(19) United States
(12) Patent Application Publication
(10) Pub. No.: US 2026/0325282 A1
(43) Pub. Date: Oct. 22, 2026

(54) ANASTOMOSIS SYSTEM

(71) Applicant: ASSISTANCE PUBLIQUE HOPITAUX DE PARIS, PARIS (FR)

(72) Inventor: Pichoy DANIAL, MAISONS-ALFORT (FR)

(21) Appl. No.: 18/880,776

(22) PCT Filed: Jun. 30, 2023
(86) PCT No.: PCT/EP2023/068106

§ 371 Date: Jan. 14, 2025

(30) Foreign Application Priority Data
Jul. 4, 2022 (FR) FR2206763

(51) Int. Cl. A61F 2/06

(52) U.S. Cl. CPC A61F 2/064; A61F 2002/065; A61F 2220/0008; A61F 2250/001

(57) ABSTRACT

An anastomosis system includes a first tube having a lateral surface, and upstream opening and a downstream opening; at least a second tube extending from the lateral surface of the first tube as far as an inlet of the second tube and communicating with the first tube; a valve mounted in the second tube; and a ligature device arranged on the circumference of the first tube downstream of the region of communication between the first tube and the second tube, the ligature device adopting a first arrangement or a second arrangement, the first arrangement closing the first tube and the second arrangement leaving said first tube open.

### TECHNICAL FIELD OF THE INVENTION

The technical field of the invention relates generally to the field of revascularisation surgery, and in particular to the field of vascular anastomosis systems used in this type of surgery.

The present invention relates to an anastomosis system.

The invention finds application in the treatment of patients suffering from vascular disease, as well as in the use of cardiac unloading pumps.

### TECHNOLOGICAL BACKGROUND OF THE INVENTION

Generally speaking, a surgical anastomosis is an artificial connection made by a surgeon between two portions of a same vessel, or between two portions of two vessels, or between two organs.

Surgical vascular anastomosis is in particular usable for repairing injured or damaged arteries and veins.

It is thus often indicated in pathologies of the aorta: aneurysm, obliterative arteriopathy of the lower limbs, aorto-occlusive injuries, etc. In these pathologies, part of the vessel is obstructed or impaired. Anastomosis then consists in redirecting the blood flow away from this part via a prosthesis, for example a synthetic tube, this tube communicating with the vessel on either side of the obstructed part.

In this configuration, the step of anastomosis into a vessel designates insertion of the end of the prosthesis into the vessel, for connection of this prosthesis with the volume of this part of the vessel.

In general, an anastomosis system refers to a prosthesis and interventional devices associated therewith.

Among vascular anastomosis systems, there are two types of system according to the way they are managed: thus there are so-called “totally endovascular” systems managed by a totally endovascular route, and vascular prostheses managed by conventional open surgery.

Systems managed by totally endovascular route do not require open surgery or laparatomy. They are introduced into the vessels by puncture through the skin or by a very short surgical approach. They are generally designated as endoprosthesis or endo-anastomosis. Theoretically, they lead to a reduction in morbidity and mortality. However, they still have a significant rate of peri-operative complications and re-operations.

Anastomosis systems managed by conventional open surgery thus remain the gold standard. They consist in approaching the anastomosis from outside the vessel.

The problem encountered is that these systems require temporary interruption of blood circulation in the vessel, during the anastomosis step in disease-free regions of the vessel. This temporary interruption of blood circulation, also referred to as clamping, has negative effects for the patient, making the treatment inapplicable to the most fragile patients, including the elderly or high-risk individuals.

The risk of complications is particularly high when clamping is supra-renal, as is the case in the treatment of obliterative arteriopathy of the lower limbs with aorto-occlusive injuries or in the treatment of aneurysms of the thoracoabdominal or abdominal aorta extended to the renal, superior mesenteric or celiac arteries. Indeed, the interruption time is long, in the order of 40-45 minutes, as the prosthesis has to be anastomosed over a great length. During this time, important organs such as the kidneys and liver are not supplied with blood and the heart is under particular pressure. This results in an increased risk of stroke, kidney failure, digestive ischaemia or even myocardial infarction.

There is therefore a need for anastomosis systems for establishing an anastomosis in a vessel or organ without interrupting blood circulation in the vessel or organ, while avoiding any blood loss.

This need is also expressed in the specific case of heart circulatory assistance systems, in particular in the case of unloading blood pump systems as described for example in application FR3112579A1. Such systems would especially benefit from access to the left atrium of the heart by anastomosis avoiding any loss of blood.

### SUMMARY OF THE INVENTION

The invention offers a solution to the problems previously discussed, by providing an anastomosis system including anti-reflux devices limiting blood loss and avoiding clamping.

To this end, one object of a first aspect of the invention is an anastomosis system which is remarkable in that it includes:
a first tube having a longitudinal axis, a side surface, an upstream opening and a downstream opening;
at least one second tube extending from the side surface of the first tube to an inlet of the second tube and communicating with the first tube by sealingly passing through the side surface, the zone of the side surface through which the second tube passes defining a communication zone between the first and second tubes;
a valve mounted in the second tube; and
a ligation device arranged on the circumference of the first tube downstream of the communication zone between the first tube and the second tube, the ligation device having a first arrangement or a second arrangement, the first arrangement closing the first tube and the second arrangement leaving said first tube open.

The system according to the invention thus includes a prosthesis (the first tube) comprising a shunt (the second tube) which makes it possible to receive interventional instruments necessary for performing an anastomosis in a vessel or an organ, this anastomosis then being performed at the upstream opening of the first tube.

More precisely, the system according to the invention makes it possible to receive medical devices used in endovascular surgery, including a crossing guide adapted to pierce the wall of the vessel or the organ and a holding guide adapted to make junction between the vessel and the organ.

By virtue of the invention, the anastomosis is made without blood loss, in a rapid, simple and reproducible manner, and with better respect for the vessel wall. In other words, the invention makes it possible to perform a minimally invasive anastomosis without blood loss. The system thus avoids resorting to clamping the vessel upstream of the anastomosis site, or else this clamping is reduced to a very short time, in the order of a few minutes, for example 5 minutes. Thus, the system can be provided for elderly or fragile patients who are generally contraindicated for conventional surgery.

More precisely, blood loss is avoided by virtue of:
a first seal, provided even before the vessel wall is crossed, by simply suturing the prosthesis around the upstream opening and the downstream opening on the vessel or organ,
a second seal, provided by the valve and the ligation device configured in its first arrangement, these preventing any blood reflux out of the system when the wall of the vessel or organ is crossed, i.e. pierced, by the crossing guide,
a third seal, provided by the holding guide at the junction between the vessel or organ and the system, the holding guide making it possible to perform an endo-anastomosis.

Advantageously, the ligation device may be a ligation clip arranged on the circumference of the first tube, the ligation clip being provided with a ligation screw.

Thus, it is possible to remotely and quickly and simply actuate tightening and untightening of the clip via the ligation screw, to configure this clip, respectively, in the first arrangement and the second arrangement.

Advantageously, the system may comprise a support guide wire extending from the inlet of the second tube through the valve to the upstream opening of the first tube, the support guide wire defining a guide path.

The support guide wire enables interventional instruments to be guided through the system in a simple and reproducible manner.

Advantageously, the system may further comprise at least one interventional instrument selected from a set of interventional instruments comprising a crossing guide adapted to pierce the wall of a vessel or an organ and a holding guide adapted to make junction between the first tube and the volume of the vessel or organ, each interventional instrument being configured to follow the guide path.

The system thus makes it possible to perform the anastomosis steps with endovascular type instruments for minimally invasive anastomosis.

Advantageously, the first tube has a first diameter and the second tube has a second diameter, it being possible for the first diameter to be equal to 9 F, 10 F, 12 F, 14 F, 16 F or 18 F, and for the second diameter to be equal to 6 F or 8 F.

Thus, the first tube is compatible with a vascular prosthesis, and the second tube is compatible with the passage of anastomosis instruments and with the fitting of a valve thereinside.

Advantageously, the holding guide cooperates with the support guide wire and comprises a stent, the stent being covered and having an expanded shape with a diameter greater than the diameter of the first tube, the expanded shape extending from the upstream end of the first tube.

In other words, the stent is expanded from inside the first tube at the upstream opening, so that its expanded shape is located as an extension of the first tube by being adjacent to the upstream opening. The stent thus makes a stable, sealed, long-term connection between the prosthesis and the vessel or organ. This is particularly advantageous when the system is applied to an organ such as the heart, as holding is stable and of good quality despite movements associated with contraction of the heart.

Furthermore, by virtue of the support guide wire, the holding guide is positioned simply, reproducibly and quickly.

One advantage of a covered stent is that it makes it possible to seal the junction between the prosthesis and the vessel, thus avoiding any blood loss.

Advantageously, the stent can be a covered balloon stent.

The balloon allows the stent to be expanded.

In a first embodiment, the first tube is a single tube, with no bifurcation (or branching or shunt).

According to one development of this first embodiment, a third tube passes through the system following the guide path, and the stent additionally comprises an attachment system.

Advantageously, the attachment system may be another stent.

This development is of particular advantage when it is applied to the inter-atrial septum of a patient's heart and used with a circulatory assistance system to unload the left side of the heart and reduce heart failure. Indeed, the third tube allows blood to circulate from the left atrium of the heart to the assistance system, avoiding peripheral access through the heart muscle to the left atrium. The advantage is that the surgical approach is much less invasive, as a thoracotomy is no longer necessary.

In addition, the attachment system makes it possible to improve holding of the anastomosis, especially with respect to cardiac movements.

In a second embodiment, the first tube includes a bifurcation into two tubes, the bifurcation defining on the first tube a straight part and a bifurcated part, the bifurcation being arranged downstream of the ligation device so that the straight part of the first tube is upstream of the ligation system and the bifurcated part is downstream of said ligation device.

Branching of the prosthesis into two downstream branches is of particular advantage when an anastomosis is performed on the abdominal aorta. Indeed, downstream, the prosthesis can be anastomosed directly on the two iliac arteries via the two bifurcations. Anastomosis is thus rapid, faster than for a conventional prosthesis where the anastomosis is termino-terminal, i.e. performed laterally, along the entire length of the prosthesis, along the aorta and the prosthesis. The advantage of the first bifurcated tube is that it avoids resorting to lengthy clamping, resulting in a lighter operation.

According to a first development of this second embodiment, the bifurcated portion of the first tube communicates with at least one fourth tube, the fourth tube extending between the side surface of the bifurcated portion of the first tube and an inlet of the fourth tube.

The fourth tube constitutes an additional branch of the prosthesis to be anastomosed on a vessel or organ. A particular advantage is found when the system is applied to the abdominal aorta. Indeed, the fourth tube can be anastomosed on one of the main arteries (renal and mesenteric arteries) supplied by the aorta. The third tube thus makes it possible to reduce the time required to perform anastomosis.

Advantageously, the bifurcated part of the first tube can communicate with a fifth, sixth and seventh tube, the fifth, sixth and seventh tubes being spaced apart on the side surface of the bifurcated part of the first tube, the spacing being longitudinal or circumferential.

Thus, the third, fifth, sixth and seventh tubes are disposed so as to be easily anastomosed on the main visceral arteries: right renal artery, left renal artery, celiac artery and superior mesenteric artery.

The invention and its different applications will be better understood upon reading the following description and upon examining the accompanying figures.

### BRIEF DESCRIPTION OF THE FIGURES

The figures are set forth by way of indicating and in no way limiting purposes of the invention.

FIG. 1 shows a schematic representation of the system according to one embodiment of the invention;

FIG. 2 shows a schematic representation of the system according to a first embodiment of the invention, with the tightening device in a tightened configuration;

FIG. 3 shows a block diagram illustrating the steps of placing the system of the invention in a vessel;

FIG. 4 shows a schematic cross-sectional representation of the system of the invention during the second step of placement in a vessel;

FIG. 5 shows a schematic cross-sectional representation of the system of the invention in the third step of placement in a vessel;

FIG. 6 shows a schematic cross-sectional representation of the system of the invention in the fourth step of placement in a vessel;

FIG. 7 shows a schematic representation of a covered balloon stent arranged on the end of an instrumental device in the system according to the invention;

FIG. 8 shows a schematic representation of a self-expandable stent arranged on the end of an instrumental device in the system according to the invention;

FIG. 9 shows a schematic cross-sectional representation of the system placed on either side of a pathological segment of a vessel;

FIG. 10 shows a schematic perspective representation of the system placed on either side of a pathological segment of a vessel;

FIG. 11 shows an unloading blood pump system for reducing heart failure connected to the left atrium of a heart by the anastomosis system of the invention;

FIG. 12 shows a schematic representation of the system according to a first development of a second embodiment of the invention;

FIG. 13 shows a schematic representation of the system according to a second development of a second embodiment of the invention;

FIG. 14 shows a schematic cross-sectional representation of the system according to one development of a first embodiment of the invention, said system being placed on a vessel or an organ;

FIG. 15 shows an unloading blood pump system for reducing heart failure connected to the left atrium of a heart via the inter-atrial septum with the system according the development of the first embodiment of the invention.

### DETAILED DESCRIPTION

The invention is concerned with an anastomosis system which avoids blood loss.

The term “anastomosis”, as used herein, will mean the surgical connection of a synthetic prosthesis, having tubular structure, into a natural structure, which can be a vessel or an organ, for the connection of this prosthesis with the volume of this vessel or this organ.

Anastomosis on the aorta and on the left atrium of the heart will be mainly discussed. Naturally, the invention can be used alternatively to connect other natural structures.

The term “endo-anastomosis” will be more particularly used when the anastomosis is obtained from inside the natural structure.

Furthermore, the terms “upstream” and “downstream” will refer to the circulation of blood flow, and the terms ‘distal’ and ‘proximal’ will refer, respectively, to an “upstream” location and a “downstream” location.

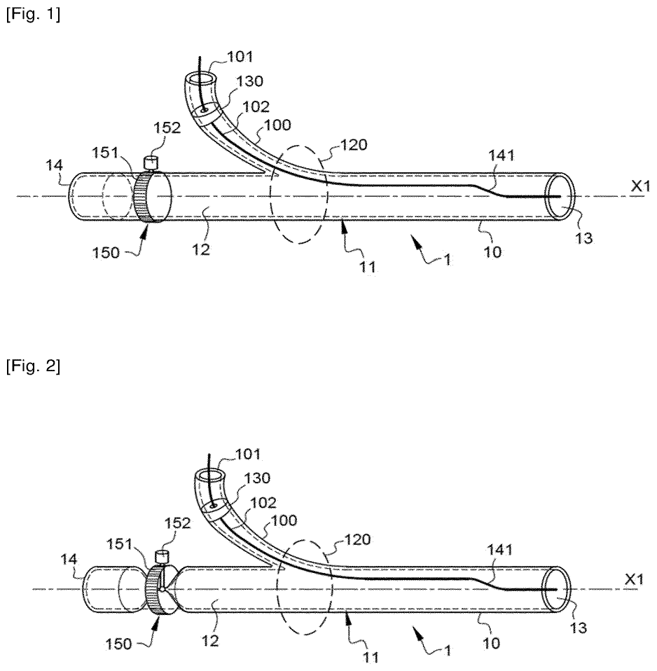

FIG. 1 shows a schematic representation of a first embodiment of the system according to the invention.

The system 1 firstly comprises a first tube 10 having a side surface 11, a longitudinal axis X1, an internal passageway 12 extending along the axis X1, and, at its ends, an upstream opening 13 and a downstream opening 14. The first tube also has a diameter that can take the following values: 9 F (3 mm), 10 F (3.33 mm), 12 F (4 mm), 14 F (4.67 mm), 16 F (5.33 mm), or 18 F (6 mm). The unit of measurement F is referred to as French (‘F’), 1 F being equal to one third of a millimetre.

The first tube 10 is of a material such as expanded polytetrafluoroethylene (ePTFE) in microporous form or polyethylene terephthalate (PET) in textile form. For example, the first tube 10 may be made of Gore Tex(R) or Dacron(R).

The system then comprises a second tube 100 extending between an inlet 101 of the second tube 100 and the side surface 11 of the first tube 10. The second tube 100 communicates with the first tube 10 through the side surface 11 of the first tube 10, i.e. the internal passageway 102 of the second tube 100 is connected to the internal passageway 12 of the first tube 10. This connection of the two internal passageways 12 and 102 defines a communication zone 120 between the first tube 10 and the second tube 100. In other words, the second tube 100 is a shunt of the first tube 10 at the side surface 11 thereof.

The second tube 100 has a diameter smaller than the diameter of the first tube 10, for example the diameter of the second tube is 2 mm (or 6 F) or 2.67 mm (or 8 F).

The second tube 100 may be of the same material as the first tube.

The second tube 100 includes a valve 130, inserted into the internal passageway 102. The valve 130 comprises in its internal volume a duct for passing a support guide wire 141 and a set of interventional instruments introduced through the inlet 101 therethrough. The valve 130 also comprises a sealing device for preventing passage of a fluid flow coming from the first tube 10 and circulating in the second tube 100.

The support guide wire 141 is introduced through the inlet 101 of the second tube 100, and then passed through the valve 130 and conveyed to the upstream opening 13 of the first tube 10 via the communication zone 120. At the upstream opening 13, the support guide wire 141 is preferably substantially centred in the centre of the upstream opening 13. Once inserted in the system, the support guide wire 141 is no longer movable. It defines a guide path for the set of interventional instruments.

Generally speaking, a guide wire includes a central core of steel or nitinol, sheaths and/or turns covering the central core, a lubricant coating covering the sheaths and/or turns, and a distal end. A guide wire further comprises radiopaque markers on the core or end. Preferably, the support guide wire 141 is stiff or extra-stiff with a softer, non-tapered distal end. Its length is preferably 180 cm and its diameter 0.889 mm (0.035 inch) but other lengths and diameters are possible, for example 300 cm for the length, 0.4572 mm (0.018 inch) or 0.3556 (0.014 inch) for the diameter.

By interventional instrument, it is meant a medical device used in endovascular surgery. Such a device generally comprises a tubular structure with or without a lumen, with a diameter generally between 0.2 (0.008 inch) and 3.33 mm (10 F), and a great length, for example greater than 40 cm.

By set of interventional instruments, it is meant a set of medical devices useful for performing anastomosis in a vessel or an organ. Such a set comprises a crossing instrument, also referred to as a crossing guide, and a holding instrument, also referred to as a holding guide.

The crossing guide is for example a crossing guide wire 140, preferably flexible and having a tapered distal end (“floppy”) configured to pierce the wall of a vessel or organ.

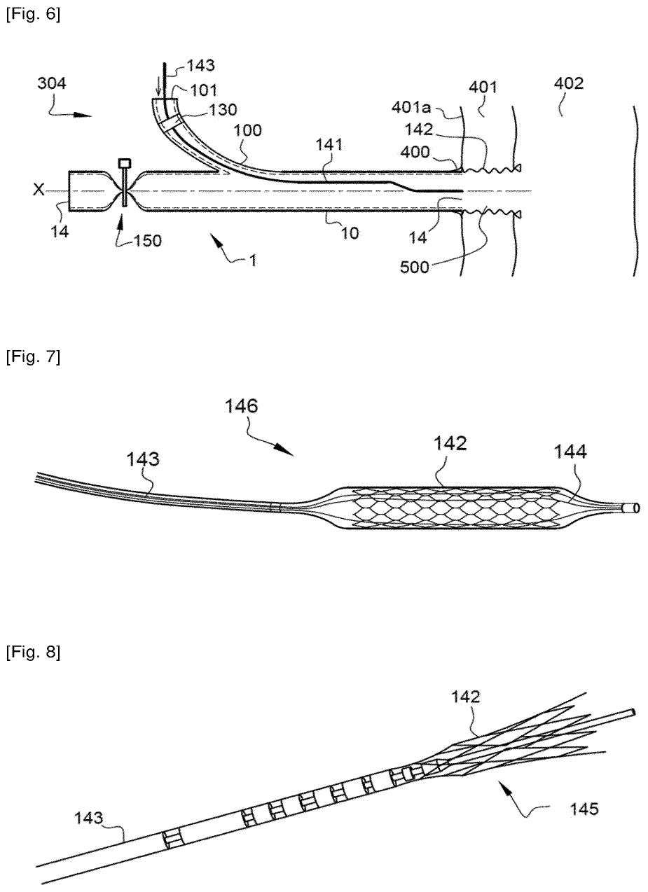

The holding guide is, for example, a balloon stent catheter 146 as illustrated in FIG. 6. Such a guide includes a stent 142 preloaded to an introduction system 143 cooperating with the support guide wire 141.

The stent 142 is comparable to a spring with a tubular shape open at its ends, which can be stretched when pressure/tension is applied thereto. By “expanded shape”, it is meant the shape that the stent 142 assumes when stretched. Preferably, the diameter of the stent, i.e. the diameter of the stent in its expanded shape, is 1 mm greater than the diameter selected for the first tube 10. For example, if the first tube 10 has a diameter equal to 10 F (3.33 mm) then the stent will have a diameter of 4.33 mm.

In this expanded shape, the stent 142 is a tube which extends from the first tube 10 from the upstream opening 13, i.e. which extends from the first tube 10 while being adjacent to the upstream opening 13.

Stent 142 is a covered stent, i.e. its surface does not allow blood to pass therethrough.

The stent 142 is made of laser-cut metal or is made from pre-welded modules. The metal is preferably a chromium-cobalt alloy but may also be steel or a nickel-titanium alloy. The stent 142 thus has good visibility under scopic inspection (X-rays), good resistance to deformation, high radial force and good stretching accuracy.

The stent 142 is preloaded at the distal end of an introducer 143 such as a catheter. A catheter is a tubular structure inserting onto the support guide wire 141. The tubular structure and the support guide wire 141 thus have a coaxial arrangement: the support guide wire 141, being fixed, acts as a guide, or rail, to guide the catheter 143 which is movable along the support guide wire 142.

The stent 142 is moreover stretched using a release system. With reference to FIG. 7 illustrating a distal portion of a holding guide 146, this release system is, for example, a balloon 144 mounted inside the stent and linked through a small tubing integrated into the introducer (not represented), the small tubing being linked opposite to the balloon to an insufflator (not represented). The balloon 144 is inflated to stretch the stent 142, leaving it in its expanded shape. The balloon 144 is then deflated and removed with the introducer 143. Another release system may be a self-expandable stent 145 as illustrated in FIG. 8.

Each interventional instrument is introduced through the inlet 101 of the second tube 100 and conveyed through the system following the guide path defined by the support guide wire 141.

The interventional instruments are movable through the system. In other words, they can slide within the system under the action of the practitioner. The instruments may also extend beyond the opening 13 in the axial direction, i.e. along the longitudinal axis X1 of the first tube 10.

Preferably, only one interventional instrument is used at a time. Thus, the catheter 143 is slid over the holding guide 146, and then the holding guide 146 is removed and the stent 142 is left in its expanded shape after removal of the introducer 143 and the balloon 144.

System 1 additionally comprises a ligation device 150 arranged on the circumference of the first tube 10 downstream of the communication zone between the first and second tubes 120. This ligation device comprises a tightening element 151 cooperating with a tightening mechanism 152 enabling the ligation element to be tightened or released. The tightening element is, for example, a ligation clip 151 disposed around the first tube 10 and the tightening mechanism is, for example, a ligation screw 152 acting on the ligation clip. Via the tightening mechanism, the ligation device assumes two arrangements: a first, tightened arrangement, illustrated in FIG. 2, which 100% ligates the first tube, thus closing it at the clip, or a second, loose, arrangement which leaves the first tube 10 open (FIG. 1).

System 1 is now described from a functional point of view.

The anastomosis is performed at the upstream opening 13 of the first tube 10 of system 1, this being in contact with the wall of a vessel or organ.

The first tube 10 thus constitutes the prosthesis, i.e. the synthetic/artificial substitute which is inserted into the vessel or organ and which serves as a duct or extension of this vessel or organ.

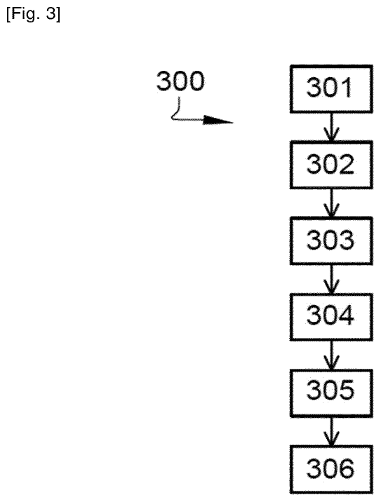

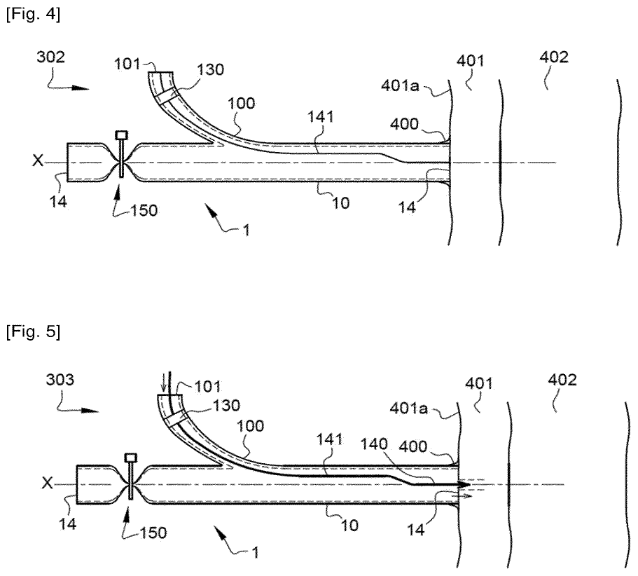

The second tube 100 serves to convey interventional instruments, these making it possible to perform an anastomosis process 300 including the anastomosis steps 301 to 306 illustrated in the diagram of FIG. 3. Steps 302, 303, 304 and 306 are in particular illustrated in FIGS. 4, 5, 6 and 9. In these figures, the system is anastomosed in a vessel, depicted by its wall 401 and its interior volume 402.

These steps relate to positioning the system 1, piercing the wall of the vessel or organ, enlarging the piercing and holding the system 1 in communication with the vessel or organ. The holding step comprises holding the dimension of the opening in the wall, holding sealing of the connection and holding both structures (vessel or organ and system 1) together.

In a first step 301, system 1 is conveyed by surgical approach to the peripheral wall 401a of a vessel or organ. The support guide wire 141 enables the system 1 to be positioned precisely on the anastomosis site by means of scopic control. For this, the support guide wire 141 is previously introduced through the inlet 101 of the second tube 100 and conveyed through the system 1 until it is in contact with the peripheral wall 401a of the vessel or organ.

In a second step 302 illustrated in FIG. 4, the first tube 10 is sutured with thread 400 to the wall 401 of the vessel 402. More precisely, the first tube 10 is sutured around its circumference at the upstream opening 13. Thus, a first seal is achieved between system 1 and the vessel without any opening having been made in the wall 401 of said vessel.

In a third step 303 illustrated in FIG. 5, the crossing guide wire 140 is introduced into the system via the inlet 101 of the second tube 100 and conveyed along the support guide wire 141 on the wall 401a of the vessel via the upstream opening 13 of the first tube 10. By virtue of a screw (not represented) and the tapered end of the crossing guide wire 140, an opening with a diameter of less than 0.3 mm is made through the wall 401 of the vessel. Blood from the vessel is then conveyed into the first tube 10 and the second tube 100. However, by virtue of the valve 120, the blood does not flow back out of the second tube 100, and by virtue of the ligation device 140 previously arranged in the first arrangement in the tightening position, blood does not flow out of the first tube 10 either. Thus, the valve 120 and the tightening device 140 make it possible to prevent blood loss without having to stop blood circulation in the vessel or organ upstream of the anastomosis site.

In a fourth step 304, the crossing guide wire is withdrawn from the system 1 through the inlet 101 of the second tube 100, and the stent 142 is expanded in the wall opening by virtue of guidance by the support guide wire 141. More precisely, with reference to FIG. 6, the stent 142 is expanded at the joint 500 between the first tube 10 and the vessel or organ, in the wall 401. The stent 142 thus expanded impacts the fragments of the wall 401 of the vessel or organ, leaving a cylindrical circulation lumen free of turbulence and therefore non-thrombogenic. Furthermore, after extraction from the introducer 143, it maintains an opening gauge at a predetermined opening, equal to the diameter of the first tube 10 plus 1 mm. In other words, the stent 142 forms a sealed duct between the vessel or organ and the system 1, this duct also having good stability and holding, despite movements associated with the human body, or handling of the system 1.

The stent 142 thus makes it possible to further reduce the risk of blood leakage. Moreover, it makes anastomosis rapid, precise, reliable and reproducible.

The stent 142 makes it possible to make an endo-anastomosis in the vessel. Indeed, the connection is made from inside the vessel 402.

In a fifth step 305, the downstream opening 14 of the first tube 10 is anastomosed according to the same method as steps 301 to 304. Both upstream and downstream openings 13 and 14 of the first tube 10 are thus each anastomosed in a vessel 402, 502. The downstream opening of the first tube 14 may also be connected to a circulatory system, for example a blood pump.

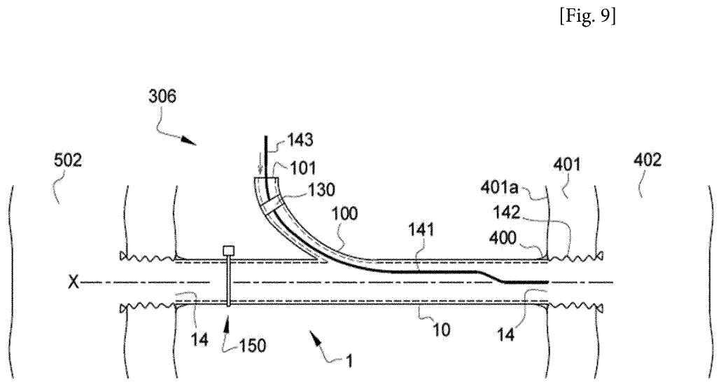

In a sixth step 306 illustrated in FIG. 9, the ligation device is in its second arrangement, so that blood freely circulates in the first tube 10.

Steps 301 to 306 are quick to perform, in the order of a few minutes. Thus, the system 1 avoids resorting to clamping.

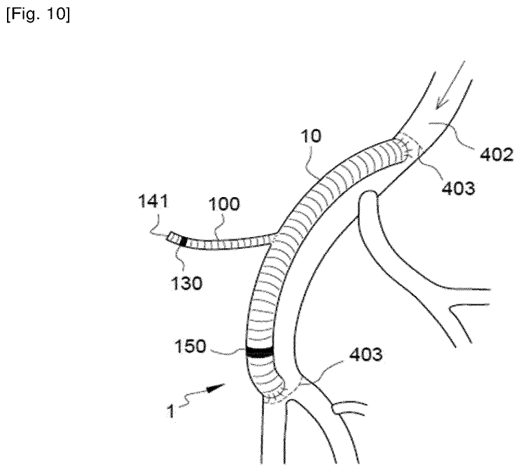

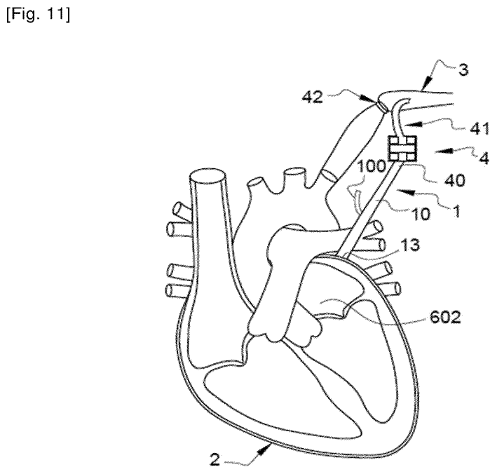

FIGS. 10 and 11 illustrate two applications of the anastomosis system 1.

With reference to FIG. 10, the first application consists of a bypass, for example a bypass of the aorta 402, with the aim of substituting a pathological segment 403 with the first tube 10 of system 1. The pathological segment 403 is, for example, an aneurysm of the aorta.

With reference to FIG. 11, the second application consists of anastomosis of the left atrium 602 of the heart to improve an unloading blood pump system 4 for treating heart failure. The system 1 is then used as a replacement for a suction cannula, making access to the heart much less hazardous.

More precisely, the unloading blood pump system 4 is mounted to unload blood from the left atrium 602 to the left subclavian artery 3.

The unloading blood pump system 4 comprises, in addition to the system 1, a reinjection cannula 41 and an unloading blood pump 40 and, in this example, a ligation member, in this case a second clip 42 which may be replaced by a lasso. The blood pump 40 comprises an inlet port 43, connected to the downstream opening 14 (referenced for example in FIG. 1) of the system 1 and an outlet port 44 connected to an inlet end 45 of the reinjection cannula 41. The upstream opening 13 of the system 1 is located in the left atrium 602 and the reinjection cannula 43 comprises a reflux end 46 opposite to the inlet end 45 located in this example in the left subclavian artery 3. The second clip 42 is clipped onto the left subclavian artery upstream of the reflux end 46, to ligate the artery. The second clip 42 or lasso can be adapted to ligate between 85% and 100% of a left subclavian artery.

The unloading blood pump 40 thus makes it possible to suck blood from the left atrium 602 through the upstream opening 13 of the system 1 and to discharge it into the subclavian artery 3 through the reflux end 46 of the reinjection cannula 41.

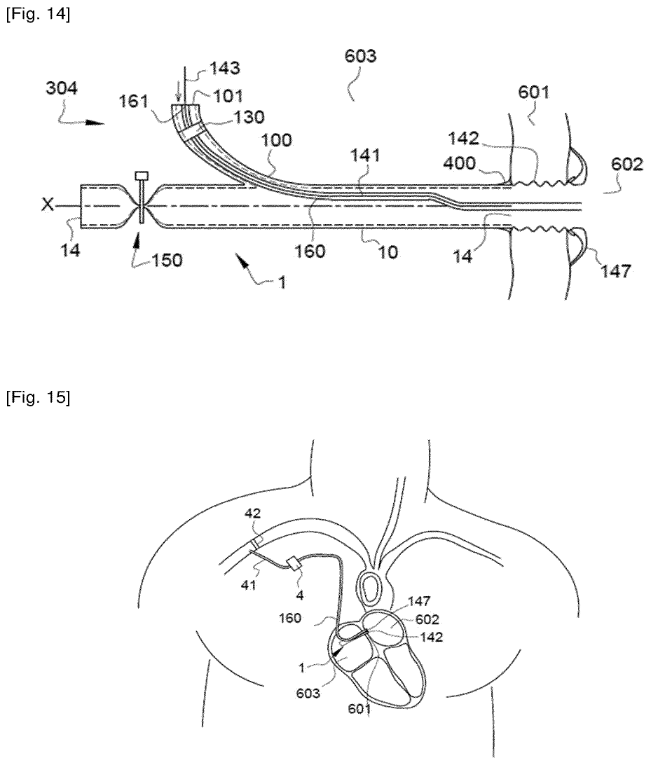

In a further development of the first embodiment illustrated in FIG. 14, the system 1 comprises a stent 142 including an attachment system 147, and a third tube 160, called an “endo-tube” because it is inserted inside the system 1. This third tube 160 is introduced through the inlet 101 of the second tube 100 and conveyed to inside the vessel or organ by following the guide path defined by the support guide wire 141.

In this development, the diameter of the first tube (10) is preferably 6 F (2 mm), 8 F (2.67 mm), 10 F (3.33 mm), 12 F (4 mm), 14 F (4.67 mm), 16 F (5.33 mm), the diameter of the second tube is preferably 6 F or 8 F, and the diameter of the stent is preferably 1 mm greater than the diameter of the first tube (10).

This development additionally finds particularly advantageous application on the inter-atrial septum 601, which is the wall separating the right atrium 601 from the left atrium 601 of the heart.

FIG. 15 illustrates system 1 used in such an application. The third tube 160 enables blood from the left atrium 602 to be circulated into an unloading blood pump 4 via the right atrium 603. The attachment system 147 makes it possible to provide a point of attachment of the system 1 to the wall of the inter-atrial septum 601 and to improve stability of holding even in the presence of cardiac movements.

By virtue of the invention, the unloading blood pump can be introduced into the patient's body below the clavicle. This approach is much less invasive than the thoracotomy required to implant the unloading pump directly into the left atrium of the heart.

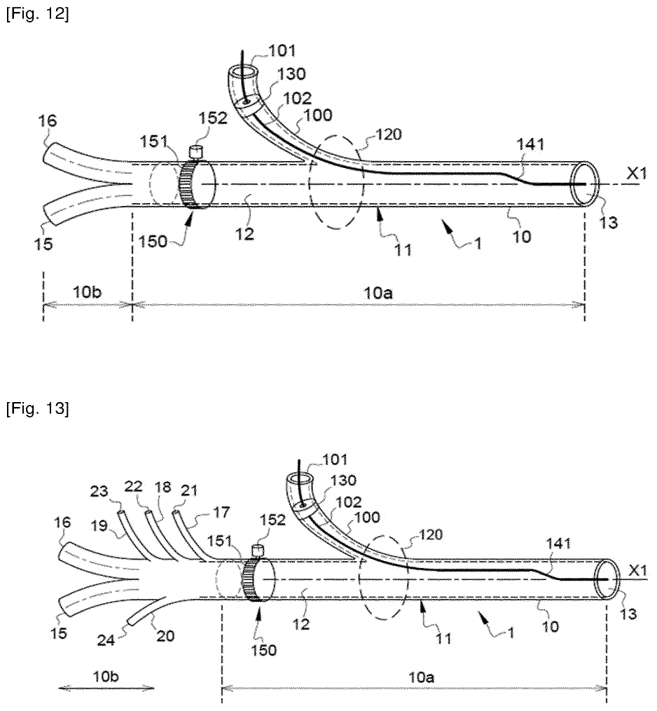

In a second embodiment illustrated in FIG. 12, the first tube 10 comprises a straight portion 10a and a bifurcated portion 10b, the bifurcation being arranged downstream of the tightening device 150. In other words, the first tube 10 divides, at the bifurcated part 10b, into two tubes 15, 16 having equal diameters, the diameter then being equal to half the diameter of the first tube 10 on its straight part 10a.

In a development of this second embodiment illustrated in FIG. 13, the first tube further comprises four side branches 17, 18, 19, 20, in the form of four tubes of the diameter 6 F (2 mm) or 8 F (2.67 mm) stretching from the side surface 11 to branch inlets 21, 22, 23 and 24. The branches are spaced apart on the side surface 11 of the first tube 10 in the axial or circumferential direction. Preferably, three branches 17, 18 and 19 are spaced apart along the axial direction, and one branch 20 is spaced apart circumferentially so as to be opposite to the three branches.

This second embodiment finds application for anastomosis of the aorta in supra-renal pathology. Indeed, this configuration makes it advantageous to rapidly anastomose inlets of the side branches with the four main arteries (renal arteries and mesenteric arteries) supplying the kidneys and liver. System 1 avoids clamping or reduces it to a few minutes (i.e. about 5 minutes), making the procedure lighter and, hence, applicable to elderly or fragile patients.

Irrespective of the embodiment, the anastomosis system 1 is implanted in the patient according to a so-called “hybrid” route based on the bases of conventional surgery and combining advantages of endo-anastomosis without clamping. In other words, system 1 allows maximum respect for the vessel wall without clamping, while ensuring better sealing at the ends of the prosthesis. The aim of system 1 is to achieve lighter surgical procedures adapted to fragile patients, which are also safer, leading to better intraoperative results and, in the longer term, shorter stays in intensive care, reduced pain and faster recovery relative to anastomosis systems of the state of the art.

1. An anastomosis system comprising:
a first tube having a longitudinal axis, a side surface, an upstream opening and a downstream opening;
at least one second tube extending from the side surface of the first tube to an inlet of the second tube and communicating with the first tube by sealingly passing through the side surface, the zone of the side surface through which the second tube passes defining a communication zone between the first and second tubes;
a valve mounted in the second tube; and
a ligation device arranged on the circumference of the first tube downstream of the communication zone between the first tube and the second tube, the ligation device having a first arrangement or a second arrangement, the first arrangement closing the first tube and the second arrangement leaving said first tube open.

2. The system according to claim 1, comprising a support guide wire extending from the inlet of the second tube to the upstream opening of the first tube by passing through the valve, the support guide wire defining a guide path.

3. The system according to claim 2, further comprising at least one interventional instrument selected from a set of interventional instruments comprising a crossing guide adapted to pierce the wall of a vessel or organ and a holding guide adapted to make junction between the first tube and the volume of the vessel or organ, each interventional instrument being configured to follow the guide path.

4. The system according to claim 3, wherein the holding guide cooperates with the support guide wire and comprises a stent, the stent being covered and having an expanded shape with a diameter greater than the diameter of the first tube, the expanded shape extending from the upstream end of the first tube.

5. The system according to claim 4, wherein the stent is a covered balloon stent.

6. The system according to claim 4, wherein a third tube passes through the system following the guide path, and in that the stent additionally comprises an attachment system.

7. The system according to claim 6, wherein the attachment system is another stent.

8. The system according to claim 1, wherein the first tube comprises a bifurcation into two tubes, the bifurcation defining on the first tube a straight portion and a bifurcated portion, the bifurcation being arranged downstream of the ligation device so that the straight portion of the first tube is upstream of the ligation system and the bifurcated portion is downstream of said ligation device.

9. The system according to claim 8, wherein the bifurcated portion of the first tube communicates with at least one fourth tube, the fourth tube extending between the side surface of the bifurcated portion of the first tube and an inlet of the fourth tube.

10. The system according to claim 1, wherein the ligation device is a ligation clip arranged on the circumference of the first tube, the ligation clip being provided with a ligation screw.
